SEQUENCE LISTING (2 sequences)

SEQ ID NO: 1 - protein, 8 aa, Synthetic construct
  source 1..8: note = Strep-tag
WSHPQFEK

SEQ ID NO: 2 - protein, 28 aa, Synthetic construct
  source 1..28: note = Twin Strep-tag
WSHPQFEKGGGSGGGSGGSAWSHPQFEK

(19) United States
(12) Patent Application Publication
(10) Pub. No.: US 2023/0349847 A1
(43) Pub. Date: Nov. 2, 2023

(54) METHOD FOR DETERMINING PROTEIN STRUCTURE USING CRYO-ELECTRON MICROSCOPY

(71) Applicant: SHENZHEN INSTITUTES OF ADVANCED TECHNOLOGY CHINESE ACADEMY OF SCIENCES, Shenzhen (CN)

(72) Inventors: Horst VOGEL, Shenzhen (CN); Zhili RAO, Shenzhen (CN); Xueying WANG, Shenzhen (CN); Xiaokang ZHANG, Shenzhen (CN); Shuguang YUAN, Shenzhen (CN); Huawei ZHANG, Shenzhen (CN)

(21) Appl. No.: 18/307,936

(22) Filed: Apr. 27, 2023

Related U.S. Application Data
(60) Provisional application No. 63/335,954, filed on Apr. 28, 2022.

(51) Int. Cl. G01N 23/2251; G01N 23/2202

(52) U.S. Cl. CPC G01N 23/2251; G01N 23/2202

(57) ABSTRACT

A method for determining a protein structure using cryo-electron microscopy, including: enabling a target protein to contain a tag; enabling a resulting target containing the tag to bind a scaffold protein to form a complex between the target protein and the scaffold protein; and performing single-particle imaging using the cryo-electron microscopy to determine a structure of the target protein in complex with the scaffold protein. The scaffold protein is any one of streptavidin, avidin, or derivatives thereof. The tag is configured for selectively binding to the scaffold protein. The tag is one selected from the group consisting of: a biotin tag, comprising a biotin; a biotinylated protein or polypeptide tag, comprising a protein sequence and a biotin covalently linked to the protein sequence; a Strep-tag; and a biotinylated or strep-tagged antibody, or antibody Fab fragment, or single-chain antibody.

### CROSS-REFERENCE TO RELATED APPLICATIONS

This application claims priority to pending U.S. Provisional Application No. 63/335,954 filed on Apr. 28, 2022, the contents of which are incorporated herein by reference in its entirety.

### SEQUENCE LISTING

The Instant Application contains a Sequence Listing which has been submitted electronically in XML format and is hereby incorporated by reference in its entirety. Said XML copy, created on Apr. 26, 2023 is named “ZYP0281US2” and is 3,268 bytes in size.

### BACKGROUND

### Technical Field

The present application relates to the technical field of structural biology, and more particularly to a method for determining a protein structure using cryo-electron microscopy.

### Description of the Related Art

The statements herein only provide background information related to the present application, and do not necessarily constitute prior art.

High-resolution three-dimensional structures of proteins deliver important information for understanding the function of these proteins (Garcia-Nafria et al, 2020). Classically protein structures are determined using X-ray crystallography and NMR spectroscopy. Depending on the sort of protein, different hurdles have to be overcome to yield a high-resolution protein structure. In the case of X-ray crystallography properly diffracting crystals have to be produced requiring large quantities of purified protein. In the case of NMR spectroscopy, the protein structure can be investigated in solution but has to be labelled in the backbone and side chains with suitable isotopes. Both structure determination techniques are very time-consuming and can be extremely expensive. This is especially the case for determining the three-dimensional structure of membrane proteins (Li et al, 2021). About one third of the human proteome are membrane proteins. Many of them play a central role for the proper function or the dysfunction of cells. Their misfunction is responsible for many serious diseases and therefore membrane proteins are the targets for most of the modern medicines. Their importance for modern drug development will increase further (Sriram and Insel, 2018).

For the determination of the three-dimensional structure of membrane proteins additional hurdles do exist as compared to water-soluble proteins. Membrane proteins first have to be isolated out of their native cellular membranes, which is typically done by dissolving the entire membrane in detergents in order to yield membrane proteins in a detergent-solubilized molecular state. Under these conditions, membrane proteins can be purified in homogenous form. For X-ray crystallography, the detergent-solubilized membrane protein has to be crystallized. In most cases membrane proteins only yield highly diffracting crystals after severe modification of their native sequence. Structure determination of membrane proteins by NMR spectroscopy are faced by additional restrictions of the size of the proteins: Only the structure of proteins of a molecular mass below 80 kDa can be routinely solved with presently available technology (Purslow et al, 2020).

Recent developments in single-particle imaging using cryo-electron microscopy (cryo-EM) have revolutionized membrane protein structural biology (Külhlbrandt, 2014; Garcia-Nafria et al, 2020). Here, many individual membrane proteins or membrane protein complexes in detergent-solubilized form or reconstituted into lipid bilayer nano-discs are imaged. From the images of millions of individual particles, it is possible to estimate the 3D structure at atomic resolution of the corresponding wild-type membrane proteins, i.e. without the need of a crystal.

Cryo-EM structures beyond 2 Å resolution have been obtained for a few large or symmetric complexes (Greber et al, 2021). However, many drug targets are neither large nor symmetric. Presently, the wider application of single-particle cryo-EM for improved resolution of membrane protein structures have been restricted by the following factors:
(i) Despite substantial technical progress, the high-resolution structure determination of such asymmetric complexes below 100-kDa molecular mass at 2.5 Å or better resolution has generally remained very challenging.
(ii) Another serious issue is protein adsorption at the air-water interface of sample preparations used for electron microscopy. This may lead to preferential protein orientation and protein denaturation reducing substantially sample quality for single-particle analysis.

### SUMMARY

In view of the above-described problems, it is an objective of the present application to provide a method for determining a protein structure using cryo-electron microscopy, which is a generic method that can solve the structure determination of target proteins of molecular mass down to at least 20 kDa.

To achieve the above objective, in accordance with one embodiment of the present application, there is provided a method for determining a protein structure using cryo-electron microscopy. The method comprises:
enabling a target protein to contain a tag;
enabling a resulting target containing the tag to bind a scaffold protein to form a complex between the target protein and the scaffold protein; and
performing single-particle imaging using cryo-electron microscopy to determine a structure of the target protein in complex with the scaffold protein.

The scaffold protein is any one of streptavidin, avidin, or derivatives thereof. The tag is configured for selectively binding to the scaffold protein. The tag is one selected from the group consisting of: a biotin tag, comprising a biotin; a biotinylated protein or polypeptide tag, comprising a protein sequence and a biotin covalently linked to the protein sequence; a Strep-tag; and a biotinylated or strep-tagged antibody, or antibody Fab fragment, or single-chain antibody.

In an embodiment, the Strep-tag is a polypeptide sequence adapted to selectively bind with streptavidin or streptactin or other derivatives thereof.

In an embodiment of the present application, in case that the tag is the biotin tag, the step of enabling the target protein to contain the tag comprises:
enabling the biotin tag to be contained in a side chain of a non-canonical amino acid, and site-specifically introducing the non-canonical amino acid into an amino acid sequence of the target protein;
or alternatively, chemically attaching the biotin tag to a specific side chain of an amino acid, such as ε-amino group of Lysine or the —SH group of Cystein, and site-specifically introducing the amino acid to the target protein;
or alternatively, chemically attaching the biotin tag to a specific glycosylation site at the N-terminal part of the target protein.

In an embodiment, the biotin tag is site-selectively attached to the target protein via chemical modification or genetic engineering.

In an embodiment of the present application, the biotinylated protein or polypeptide tag is one selected from the group consisting of:
a self-labeling protein tag, which allows site-specific covalent attachment of a biotin residue to a respective tag protein;
an acyl carrier protein tag (ACP-tag) or a peptidyl carrier protein tag (PCP-tag); and
an Avi-tag, adapted to be fused to the N-terminus, the C-terminus, or an exposed loop region of the target protein, and to be covalently attached to the biotin using Escherichia coli biotinylase BirA.

In an embodiment of the present application, the ACP-tag or the PCP-tag and the biotin are covalently attached enzymatically by Sfp- and AcpS-PPTases, respectively, using biotin derivates of CoA as substrates.

In an embodiment of the present application, the self-labeling protein tag is a Snap-tag, a Clip-tag, or a Halo-tag.

In an embodiment of the present application, the target protein has a molecular mass preferentially between 50 kDa and 20 kDa or less. In an embodiment of the present application, the target protein has a molecular mass down to at least 33 kDa. In an embodiment of the present application, the target protein has a molecular mass down to at least 20 kDa.

In an embodiment of the present application, the target protein is a water-soluble protein or a membrane protein.

In an embodiment of the present application, in the case that the target protein is a G protein-coupled receptor (GPCR), the biotin tag or the biotinylated protein or polypeptide tag is inserted to the N-terminus, the C-terminus, one of extracellular loops (that is, E1, E2, or E3), or one of intracellular loops (that is, I1, I2, I3, or I4) of the GPCR.

In an embodiment of the present application, in the case that the target protein is a G protein-coupled receptor (GPCR), an anticalin or an Ig type or single-chain antibody is selectively bound to the N-terminus, the C-terminus, one of extracellular loops (E1, E2, or E3), or one of intracellular loops (I1, I2, I3, or I4) of the GPCR.

In an embodiment of the present application, the biotin tag or the biotinylated protein or polypeptide tag or a Strep-tag is fused into an intracellular loop I4 of the GPCR, so as to stabilize a bound streptavidin or streptactin in a rigid structure.

In an embodiment of the present application, the biotin tag or the biotinylated protein or polypeptide tag or the Strep-tag is fused into one of extracellular loops (E1, E2, or E3) or one of intracellular loops (I1, I2, I3, or I4) of the GPCR, so as to stabilize a bound streptavidin or streptactin in a rigid structure.

In an embodiment of the present application, in the case that the target protein is a complex of a prototypical GPCR and an intracellular signaling protein, the tag is introduced by adopting any one of the following manners, so as to enable binding of the complex to the streptavidin or the derivative thereof as the scaffold protein:
1) inserting the biotin or the biotinylated protein or polypeptide tag to an N-terminus, a C-terminus, one of extracellular loops, or one of intracellular loops of the prototypical GPCR;
2) selectively binding an anticalin or an Ig type or single-chain antibody to the N-terminus, the C-terminus, one of extracellular loops, or one of intracellular loops of the prototypical GPCR;
3) fusing the biotin tag or the biotinylated protein or polypeptide tag to the intracellular signaling protein sequence;
4) selectively binding a biotinylated antibody to the intracellular signaling protein; or
5) selectively binding a biotinylated anticalin to the intracellular signaling protein.

In an embodiment of the present application, the intracellular signaling protein is any one selected from the following:
a heterotrimeric G-protein or a mini-G-protein, both of which are adapted to bind to an agonist-activated GPCR;
a G-protein-coupled receptor kinase (GRK), adapted to bind to and phosphorylates an active GPCR;
an arrestin, adapted to bind to a phosphorylated GPCR; and
a peptide sequence, mimicking a region of the G-protein or the arrestin for receptor binding.

In an embodiment of the present application, in the case that the target protein is a neurokinin 1 receptor (NK1R), the tag is a biotinylated Halo-tag, the scaffold protein is streptavidin; a tetrameric NK1R is assembled on the streptavidin via the biotinylated Halo-tag, whereby forming a complex SA(HaloTag-NK1R)ₙ, in which, n is an integer ranged between 1 and 4, for example, n is 1, or 2, or 3, or 4. The complex SA(HaloTag-NK1R)ₙ is suitable for determining a structure of the NK1R by the cryo-EM.

In an embodiment of the present application, the biotinylated Halo-tag is inserted into a third intracellular loop IL3 of the NK1R, and a sequence of between amino acid 227 and 237 is removed from the third intracellular loop.

In an embodiment of the present application, before the step of enabling the target protein to contain the tag, the method further comprises:
synthesizing a HaloTag-PEG4-biotin ligand, and
forming a stable ester bond between the HaloTag-PEG4-biotin ligand and a HaloTag protein whereby forming a biotinylated Halo-Tag.

In an embodiment of the present application, the HaloTag-PEG4-biotin ligand has a molecular formula of C₃₁H₅₇ClN₄O₉S, a molecular mass of 697, and the following chemical structure:

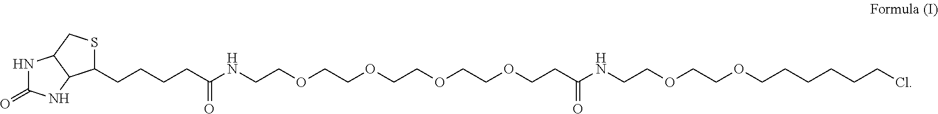 [SMILES: O=C(CCCCC1SCC2NC(=O)NC21)NCCOCCOCCOCCOCCC(=O)NCCOCCOCCCCCCCl]

A terminal —Cl of the HaloTag-PEG4-biotin ligand is configured to be displaced in a nucleophilic reaction by an Asp106 of the HaloTag protein to form the stable ester bond, whereby forming the biotinylated Halo-Tag.

In an embodiment of the present application, a PEG4 spacer in the HaloTag-PEG4-biotin ligand is further shortened or enlarged to obtain an optimal structural rigidity between the scaffold protein and the target protein.

In an embodiment of the present application, before the step of enabling the target protein to contain the tag, the method further comprises: molecular modeling, during which, a proper position in the target protein suitable for inserting the biotin tag or the biotinylated protein or polypeptide tag or the Strep-tag is found and adjusted, to obtain an optimal structural rigidity between the scaffold protein and the target protein.

In an embodiment of the present application, during the molecular modeling, spacer amino acids are inserted into an amino acid sequence of the target protein, or flexible amino acid sequences which are functionally not relevant are removed from the amino acid sequence of the target protein.

Advantages of the method for determining a protein structure using cryo-electron microscopy according to embodiments of the present application are summarized as follows:

In the method of the present application, a target protein is enabled to contain a tag. A resulting target protein containing the tag is allowed to bind with a scaffold protein to form a complex. Then single-particle imaging is performed using cryo-electron microscopy to determine the structure of the target protein. The tag is capable of selectively binding to the scaffold protein. In this way, the method is generic for structure determination of many kinds of soluble proteins or membrane proteins. Thereby, the target protein may not be restricted to symmetric structures and large molecular mass. In contrast, the method of the present application can realize the structure determination of target proteins of molecular mass down to at least 20 kDa, and the result of the determination is not affected by the structure symmetry of the target protein.

It is another objective of the present application to provide use of HaloTag-PEG4-biotin ligand in the above-mentioned method for determining a protein structure using cryo-electron microscopy. The HaloTag-PEG4-biotin ligand has a molecular formula of C₃₁H₅₇ClN₄O₉S, a molecular mass of 697, and the following chemical structure:

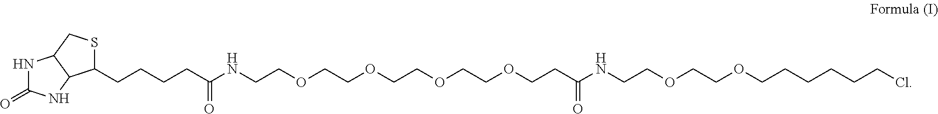 [SMILES: O=C(CCCCC1SCC2NC(=O)NC21)NCCOCCOCCOCCOCCC(=O)NCCOCCOCCCCCCCl]

In an embodiment of the present application, a terminal —Cl of the HaloTag-PEG4-biotin ligand is configured to be displaced in a nucleophilic reaction by an Asp106 of a HaloTag protein to form a stable ester bond, whereby forming a biotinylated Halo-Tag.

Advantages of the HaloTag-PEG4-biotin ligand according to embodiments of the present application are summarized as follows:

The terminal —Cl of the HaloTag-PEG4-biotin ligand can be displaced in a nucleophilic reaction by the Asp106 of the HaloTag protein to form a stable ester bond, so as to form the biotinylated Halo-Tag. The biotinylated Halo-Tag is capable of connecting the target proteins to the scaffold protein to form a protein complex, which is suitable to perform the single particle cryo-EM to determine the structure of the target protein. The length of the PEG4 spacer in the HaloTag-PEG4-biotin ligand might be changed to minimize flexibility between HaloTag protein and streptavidin scaffold protein.

### BRIEF DESCRIPTION OF THE DRAWINGS

The present application is described hereinbelow with reference to the accompanying drawings, in which:

FIGS. 1A-1C show tetramer formation of target proteins or protein complexes on a scaffold protein for single-particle cryo-electron microcopy. In particular, FIG. 1A shows a schematic representation of a prototypical G-protein-coupled receptor (GPCR) composed of seven transmembrane helices (rectangles 1, 2, . . . 7) and helix 8 (rectangle 8) at the intracellular C-terminus. An extracellular N-terminus comprises a biotinylated tag; FIG. 1B shows a schematic representation of a prototypical GPCR in complex with an intracellular signaling protein; and FIG. 1C shows a schematic representation of a scaffold protein comprising one protein of interest (POI) specifically bound via its tag to each of the four scaffold binding sites.

FIG. 2A shows a structural model of SA-(NK1R-halotag)₄, comprising tetrameric streptavidin, SA (center), and four bound molecules of biotinylated HaloTag proteins. The structural model was obtained by computer modeling.

FIG. 2B shows intrinsic mobility of the protein complex calculated from anisotropic network model (ANM) analysis.

FIG. 3A shows a structural model of SA(HaloTag)₄, comprising tetrameric streptavidin, SA, (center, orange color) and four bound molecules of biotinylated HaloTag proteins (green color), one HaloTag protein per SA subunit.

FIG. 3B shows an intrinsic mobility of complex calculated from ANM analysis.

FIG. 4A shows purified Halotag-PEG4-biotin ligand analyzed by HPLC: the optical absorption wavelength was 210 nm and the retention time 10 min.

FIG. 4B shows LC-MS of Halotag-PEG4-biotin ligand. MS: m/z calcd. for C₃₁H₅₈ClN₄O₉S⁺: 697.36 (100%), 698.36 (36.6%), 699.36 (41%), 719.3 (12.13%), 720.3 (4.41%).

FIG. 5A shows a size exclusion chromatography of streptavidin-halotag protein complex.

FIG. 5B shows SDS-PAGE of streptavidin tetramer (streptavidin), halotag protein (halotag), size exclusion chromatography fraction 1 and fraction 2 (FIG. 5A).

FIG. 6 shows an electron micrograph of negative stained preparation of the streptavidin-halotag protein complex isolated by size exclusion chromatography (FIG. 5A, fraction 1).

FIG. 7A shows a representative cryo-electron micrograph of streptavidin-halotag protein complexes (cryo-electron microscope operated at 300 kV. Scale bar: 50 nm). FIG. 7B shows representative 2D class-averaged images of the streptavidin-halotag protein complex.

FIGS. 8A-8B show resolution estimation of cryo-EM map of streptavidin-halotag protein complex. Particularly, FIG. 8A show Fourier shell correlation (FSC) plots indicating corrected resolutions as 3.6 Å; FIG. 8B is a 3D map of streptavidin-halotag protein complex after non-uniform refinement.

FIG. 9A shows a size exclusion chromatography of streptavidin-NK1R-halotag complex; and

FIG. 9B shows SDS-PAGE of streptavidin-NK1R-halotag complex, NK1R-Halotag, corresponding to size exclusion chromatography fraction 1 to fraction 4 (FIG. 9A).

### DETAILED DESCRIPTION OF THE EMBODIMENTS

To further illustrate the present application, experiments detailing a method for determining a protein structure using cryo-electron microscopy are described below. It should be noted that the following examples are intended to describe and not to limit the present application

### Definitions

Where an indefinite or definite article is used when referring to a singular noun, such as “a” or “an”, “the”, unless otherwise specified, include the plural form of the noun. When the term “comprising” is used in this specification and claims, it does not exclude other elements or steps. In addition, the terms first, second, third, etc. in the specification and claims are used to distinguish similar elements, and not necessarily used to describe order or time sequence. It should be understood that the terms so used are interchangeable under appropriate circumstances, and that the embodiments of the invention described herein can operate in other orders than described or illustrated herein. The following terms or definitions are provided only to help understand the present invention. Unless specifically defined herein, all terms used herein have the same meaning as understood by those skilled in the art of the present invention.

As used herein, the term “target protein” or “protein of interest” refers to a protein whose structure is to be determined.

“scaffold protein” used herein refers to proteins having multiple protein binding domains that bind specifically a tag or a modified tag attached to the protein whose structure is to be determined.

The terms “protein”, “polypeptide” and “peptide” are further used interchangeably herein to refer to polymers of amino acid residues and their variants and synthetic analogs.

Biotin is hexahydro-2-oxo-1H-thieno(3,4-d)imidazole-4-pentanoic acid. It is also known as vitamin H and coenzyme R, and is a water-soluble vitamin and also belongs to the vitamin B family, B7. It is a necessary substance for the synthesis of vitamin C and an indispensable substance for the normal metabolism of fat and protein.

Streptavidin is a protein complex made up of four identical protein subunits. The tetramer has a molecular mass of 52 kDa protein and can be purified from the bacterium Streptomyces avidinii or can be produced by heterologous protein expression in E.coli. Streptavidin homo-tetramers have an extraordinarily high affinity for biotin (also known as vitamin B7 or vitamin H). With a dissociation constant (Kd) on the order of ≈10⁻¹⁴ mol/L, the binding of biotin to streptavidin is one of the strongest non-covalent interactions known in nature. Streptavidin is used extensively in molecular biology, bioanalysis and biotechnology due to the streptavidin-biotin complex's resistance to organic solvents, denaturants (e.g. guanidinium chloride), detergents (e.g. SDS, Triton X-100), proteolytic enzymes, and extremes of temperature and pH.

Water soluble proteins of interest in the present context having a molecular mass below 50 kDa include enzymes, transcription factors and transport proteins, are found free in cellular compartments such as the cytoplasm, nucleus, or endoplasmic reticulum.

Integral membrane proteins, briefly mentioned herein as “membrane proteins”, are proteins that are embedded among the lipids that make up the bilayer structure of cell membranes. The membrane proteins perform specific tasks that are essential for the proper functioning of the cell. These include translocation of molecules and ions into and out of the cell, detecting extracellular signals and transmitting them into cells. The extracellular sites of membrane proteins are often glycosylated making the cell recognizable to other cells.

G protein-coupled receptors (GPCRs) are integral membrane proteins that transmit physical and chemical signals from the extracellular space into the cell. The chemical signals recognized by GPCRs range from small molecules like neurotransmitters to hormones and proteins. There are about 800 representatives in the human proteome GPCRs establish the largest family of membrane proteins. There are five different classes of GPCRs in humans. The rhodopsin-like class A contains the largest number of GPCRs with about 700 representatives. About half of them are olfactory receptors. The molecular mass of typical class A GPCRs are in the range of 30 to 40 kDa. Each receptor contains a transmembrane domain composed of seven α-helices. The extracellular N-terminus is often glycosylated. Specific conserved glycosylation sites can be selectively biotinylated by a one-step chemical reaction. The intramembrane C-terminus contains often an α-helix which is attached via lipid anchors to the intracellular side of the plasma membrane. GPCRs are among the most important targets for small molecule drugs.

Neurokinin 1 receptor (NK1R) is the main receptor for the tachykinin peptides such as substance P. NK1R belongs to class A GPCRs. It couples to Gq and Gs-protein signaling pathway.

As used herein, the term “fusion”, and interchangeably used herein as “conjugating”, “linking”, especially referring to “genetic fusion”, such as by recombinant DNA technology, and referring to “Chemical and/or enzymatic binding” leading to stable covalent linkage.

As used herein, the term “protein complex” or “complex” or “assembled protein” refers to a group of two or more bound macromolecules, at least one of which is a protein.

The term “binding” means any interaction, whether it is direct or indirect. Direct interaction suggests contact between binding partners. Indirect interaction means any interaction whereby the interaction partner interacts in a complex of more than two molecules.

As used herein, the term “antibody” refers to an immunoglobulin G (IgG) molecule or a molecule containing an immunoglobulin (Ig) domain that specifically binds to an antigen. The antibody may be a whole immunoglobulin from a natural source or from a recombinant source and may be an immunoreactive part of a whole immunoglobulin. IgG antibodies are generally made of four peptide chains.

As used herein, the term “anticalin” refers to proteins which are engineered lipocalins with novel, antibody-like binding functions.

As used herein, the term “label” or “tag” refers to a detectable probe that allows the detection, visualization, and/or separation, purification, and/or immobilization of the (poly)peptide or protein described herein that has been isolated or purified. It is intended to include any marker/label known in the art for these purposes.

The term “wild type” refers to a gene or gene product isolated from a naturally occurring source. The wild-type gene is the most commonly observed gene in the population, so the “normal” or “wild-type” form of the gene is arbitrarily designed. On the contrary, the terms “modified”, “mutant”, “derivative”, or “variant” refer to the sequence, post-translational modification and/or functional characteristics (i.e. changed characteristics) that are displayed when compared with the wild-type gene or gene product or modified gene or gene product.

The terms “vector”, “vector construct”, “expression vector” or “gene transfer vector” as used herein are intended to refer to a nucleic acid molecule capable of transporting another nucleic acid molecule linked to it, and includes any known to those skilled in the art vectors, including any suitable type, including but not limited to plasmid vectors, cosmid vectors, phage vectors (such as lambda phage), viral vectors (such as adenovirus, AAV or baculovirus vectors) or artificial chromosome vectors (such as bacterial artificial chromosomes) (BAC), yeast artificial chromosome (YAC) or P1 artificial chromosome (PAC).

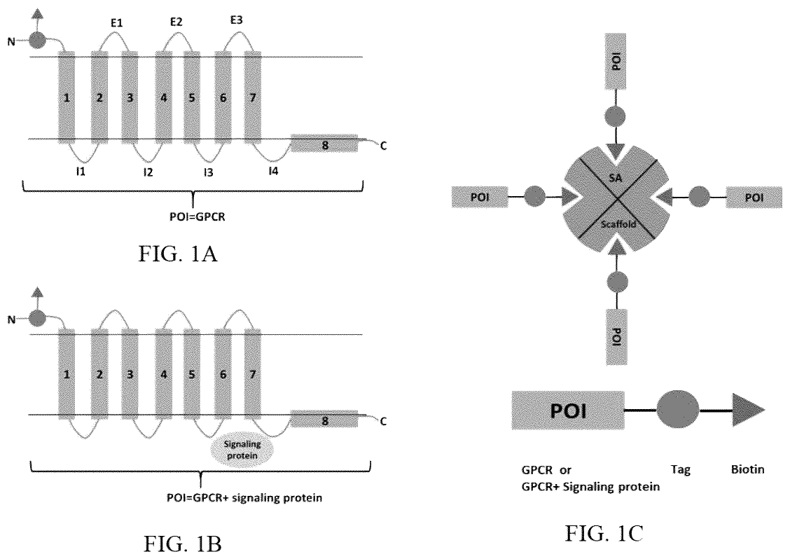

Here we present a novel technique to solve the structure of target proteins of molecular mass down to at least 20 kDa. It is generic, i.e. it can be identically used for many different target proteins. The target protein for structure determination comprises a tag which selectively binds to streptavidin, avidin or modified versions thereof (FIG. 1). The tags of interest are typically proteins or short peptide sequences to which biotin can be covalently attached. Alternatively, so-called strep-tags can be used which are short peptide sequences capable to bind selectively to streptavidin or modified versions of streptavidin such as streptactin or streptactin-XT. The different tags of interest are summarized in Table 1.

TABLE 1

| | | |
|---|---|---|
| Biotin attached to non-natural | Non-natural | Kim, et al (2013) |
| amino acids by genetic | amino acid site- | |
| engineering of target protein | specifically introduced | |
| | into the amino acid | |
| | sequence of the | |
| | target protein | |
| Biotin directly attached to | Chemical modification | Bieri et al, (1999) |
| glycosylation sites of | of specific glycosylation | |
| target protein. | sites at N-terminal | |
| | part of target | |
| | protein. | |
| Snap-tag | Expression vectors of | Reymond et al, (2011) |
| Self-labeling protein tag of | Snap-tag protein and | https://international.neb.com |
| 182 residues (19 kDa). | of Snap-tag biotin-label | |
| Fusion with POI. | are commercially available. | |
| Engineered O6-alkylguanine-DNA | SNAP-tag ® is a | |
| alkyltransferase. | registered trademark | |
| Covalent, site-selective attachment | of New England Biolabs, | |
| of biotin to Snap-tag. | Inc. | |
| Clip-tag | Expression vectors of | Reymond et al, (2011) |
| Engineered derivative of Snap-tag. | Clip-tag protein and | |
| Fusion with POI. | of Clip-tag biotin-label | |
| Covalent, site-selective attachment | are commercially | |
| of biotin to Clip-tag. | available. | |
| | CLIP-tag ™M is a | |
| | trademark of New England | |
| | Biolabs, Inc. | |
| Halo-tag | | Los et al, 2008 |
| Self-labeling protein tag of 297 | Expression vectors of | https://www.promegaconnections.com |
| residues (33 kDa). | Halo-tag protein and | |
| Engineered bacterial haloalkene | of Halo-tag biotin-label | |
| dehalogenase. | are commercially | |
| Fusion with POI. | available. | |
| Synthetic substrate contains a | | |
| reactive chloroalkane linker | | |
| bound to a functional group. | | |
| ACP- and PCP-tags | Expression vectors and | George et al, 2004 |
| Small protein tags based on | biotin-label are | Vivero-Pol et al, 2005 |
| acyl carrier protein (ACP) | commercially available. | https://international.neb.com |
| or peptidyl carrier protein | | |
| (PCP). | | |
| Labelled site-specifically | | |
| with biotin using AcpS- and | | |
| Sfp- phosphopantetheinyl | | |
| transferases | | |
| Substrates are derivatives | | |
| of Coenzyme A (CoA). | | |
| Fusion with POI. | | |
| A1 and S6 peptide tags | Expression vectors and | Zhou et al, 2007 |
| The 12-residue peptide tags | biotin-label are | https://international.neb.com |
| can be fused to POI and | commercially available. | |
| labelled site-specifically | | |
| with biotin by Sfp- and | | |
| AcpS-PPTases, respectively, | | |
| using biotin derivates of | | |
| CoA as substrates. | | |
| Strep-tag or twin strep-tag | Expression vectors for | https://en.wikipedia.org/wiki/Strep-tag |
| 8 or 28-amino acids peptide, | strep-tag, twin strep- | https://www.iba-lifesciences.com |
| H₂N—WSHPQFEK—COOH | tag and streptactin, | |
| (SEQ ID NO:1) or | streptactin-XT are | |
| WSHPQFEKGGGSGGGSGGSAWSHPQFEK | commercially available. | |
| (SEQ ID NO: 2) can be fused | | |
| to a POI to act as a tag. The | | |
| strep-tag can bind strongly to | | |
| one and the twin strep-tag to | | |
| two of the four identical | | |
| binding sites of streptactin | | |
| or strep-tactin-XT, a modified | | |
| version of streptavidin. In | | |
| total, four strep-tags fused | | |
| to the corresponding target | | |
| protein can bind to one | | |
| streptavidin or streptactin | | |
| and two twin strep-tags fused | | |
| to the corresponding target | | |
| protein can bind to one | | |
| streptactin or strep-tactin-XT | | |
| molecule. | | |
| Anticalins, Antibodies, Fab | Anticalins or Antibodies | Gebauer and Skerra, 2020. |
| fragments, Single-chain | targeting GPCRs | Manglik et al, 2017 |
| antibodies (nanobodies) | Anticalins or Antibodies | a) scFv16 |
| Bind with high affinity | targeting GPCR/G- | Fab30: Lee et al, 2020; |
| selected native or engineered | protein complexes | Baidya et al, 2020 |
| regions in POI. These antibody | Anticalins or Antibodies | b) Arrestin-1 monoclonal |
| binding regions on the POI | targeting arrestins | antibody (ZA005) |
| can be located on the GPCR or | Antibodies targeting | Fab1, Fab6: Chen et al, 2021 |
| on the signaling protein. | GRKs | |
| The antibodies and antibody | | |
| fragments contain at selected | | |
| sites covalently attached biotin | | |
| to enable the binding to | | |
| streptavidin. | | |

In general, the tags can be distinguished in two groups summarized in Table 1.

(1) Biotin, directly attached to target protein or biotinylated protein tag or biotinylated polypeptide tag fused to the sequence of the target protein-of-interest.

The fusion protein of interest comprises one of the following tags:
(i) a biotin contained in the side chain of a non-canonical amino acid introduced site-specifically into the amino acid sequence of the target protein. (Kim et al, 2013).
(ii) a biotin chemically attached to a specific glycosylation site at the N-terminal part of the target protein (Bieri et al, 1999). More specifically, a glycosylation consensus site close to the extracellular N terminus is conserved among sequenced GPCRs, and glycosylation has been confirmed for several receptors. This feature can be exploited using carbohydrate-specific chemistry for biotinylation, thereby confining the biotin tag to the extracellular domain of the receptor.
(iii) A self-labeling protein tag such as Snap-tag, Clip-tag or Halo-tag which allow the site-specific covalent attachment of a biotin residue to the respective protein tag (Los et al, 2008; Reymond et al, 2011).
(iv) An acyl carrier protein (ACP) or peptidyl carrier protein (PCP) tag which can be labelled with biotin site-specifically using Sfp and AcpS phosphopantetheinyl transferases (PPTases), respectively (George et al, 2004; Vivero-Pol et al, 2005). The ACP and PCP tags can be replaced by 12-residue peptide tags A1 and S6 which can be labelled with biotin site-specifically at the fusion proteins by Sfp- and AcpS-PPTases, respectively (Zhou et al, 2007).
(v) A strep tag, composed of a 8-amino acids peptide (SEQ ID NO: 1: WSHPQFEK), can be used (https://en.wikipedia.org/wiki/Strep-tag; Schmidt & Skerra, 2007), whose sequence binds strongly to one of the four identical binding sites of Strep-Tactin, a modified version of streptavidin. The strep-tag can also bind to streptavidin but with lower affinity as compared to Strep-Tactin. In total, four strep-tags fused to the corresponding target protein will bind to one Strep-Tactin or streptavidin molecule. Alternatively, a twin-strep-tag can be used (SEQ ID NO: 2: WSHPQFEKGGGSGGGSGGSAWSHPQFEK) which binds with higher affinity to Strep-Tactin and with sub-nanomolar affinity to Strep-Tactin-XT, another modified version of streptavidin. The twin-strep-tag can also bind to Strep-Tactin or streptavidin but with lower affinity as compared to Strep-Tactin-XT. Here, one twin-strep-tag sequence binds to two subunits of Strep-Tactin-XT, Strep-Tactin or streptavidin (https://www.iba-lifesciences.com; Palmer I et al, (2007)) i.e. in this case only two twin-strep-tag sequences can bind to one streptavidin, Strep-Tactin or Strep-Tactin-XT.
(vi) Another alternative is the 15-amino acid Avitag which can be fused to the N- or C-terminus or an exposed loop region of a target protein and to which a biotin can be covalently attached using the Escherichia coli biotinylase BirA (Fairhead & Howarth, 2015).

The above-mentioned tags offer the additional advantage to be used for affinity purification of the target protein (https://en.wikipedia.org/wiki/Strep-tag; Lin et al, 2020).

(2) Biotinylated or strep-tagged antibodies or antibody Fab fragments or single-chain antibodies (nanobodies) or anticalins (Gebauer & Skerra, 2020) binding selectively to target protein-of-interest (POI).

In this approach the biotin or the strep-tag sequence is covalently attached to an IgG-type antibody, antibody fragment or nanobody which binds with high affinity and selectively to a native or genetically introduced sequence or structural motive in the POI. This approach is especially of interest if a native, unmodified POI should be assembled to the scaffold protein streptavidin or its derivatives.

FIG. 1A-1C depict the possibilities to position the biotinylated tags and strep-tags in the case of either water-soluble proteins or membrane proteins such as GPCRs. GPCRs are the largest class of human membrane proteins and are among the most important drug targets (Zhou et al, 2019; Sriram & Insel, 2018). As most of the GPCRs comprise an S-palmitoylation at a certain cysteine residue in the C-terminal region (Qanbar & Bouvier, 2003; Adachi et al, 2019; Patwardhan et al, 2021), the intracellular loop 14 between transmembrane helix 7 and the S-palmitoylation site is of special interest to place the biotin/Strep-tag as it might stabilize the bound streptavidin/streptactin in a rigid structure. Similarly, biotin/Strep-tag (or biotinylated A1 and S6 tags) placed in one of the extracellular loops E1, E2, E3 or one of the intracellular loops I1, I2, I3, I4 might keep the bound streptavidin/streptactin in a rigid structure, which would facilitate structure elucidation of the target GPCR.

FIG. 1A shows a schematic representation of a prototypical G-protein-coupled receptor (GPCR) composed of seven transmembrane helices (rectangles 1, 2, . . . 7) and helix 8 (rectangle 8) at the intracellular C-terminus. The C-terminal part of the GPCR might be attached to the intracellular side of the lipid bilayer nanodisc via a lipid anchor. In the representation shown, the extracellular N-terminus comprises a single tag (for example, a biotinylated tag) which is capable to bind selectively either directly or after posttranslational modification to one of the four distinct binding sites of the scaffold protein (as shown in FIG. 1C). Typically, the target GPCR comprises one single protein- or peptide-tag, which might be inserted either on the N-terminus, on the C-terminus or into one of the extracellular (E1, E2, E3) or intracellular I2, I3, I4) loops, respectively. Alternatively, IgG type or single-chain antibodies targeting selectively the extracellular or the intracellular side of a GPCR might act as biotinylated tags to bind to a streptavidin or streptavidin-related scaffold. A list of potential protein- and peptide-tags is given in Table 1.

FIG. 1B shows a schematic representation of a prototypical GPCR in complex with an intracellular signaling protein, such as: (i) a heterotrimeric G-protein or a mini-G-protein which both can bind to an agonist-activated GPCR; (ii) a G-protein-coupled receptor kinase (GRK) which binds to and phosphorylates an active GPCR; (iii) an arrestin which binds to a phosphorylated GPCR; and (iv) peptide sequences which mimic regions of G-protein or arrestin for receptor binding.

There are different approaches to introduce tags to such GPCR/signaling-protein complexes to enable binding of these complexes to the streptavidin or streptavidin-like scaffold protein: (i) using GPCRs comprising tags as described in (FIG. 1A); (ii) attaching a particular tag to either of the signaling proteins or signaling peptide sequences, herein, in some embodiments, the particular tag is a biotinylated protein or polypeptide tag fused to a sequence of the target protein; and (iii) using biotinylated anticalins or antibodies targeting the signaling protein bound to GPCR. FIG. 1C shows a schematic representation of a scaffold protein comprising one protein of interest (POI) specifically bound via its tag (for example, the biotinylated tag) to each of the four scaffold binding sites. The scaffold protein might be streptavidin or avidin which specifically binds a biotin of the tag. Alternatively, the 11-aminoacid strep-tag can be used which binds selectively to the scaffold protein streptactin, a modified version of streptavidin. The POI corresponds either to a GPCR with an attached tag as described in (FIG. 1A), or to a GPCR/signaling-protein complex with an attached tag as described in (FIG. 1B).

The feasibility of the new approach to use a GPCR assembled via halotag on a streptavidin template for elucidating the structure of the corresponding GPCR by single-particle cryo-EM is demonstrated using the neurokinin 1 receptor (NK1R) as a prototypical GPCR. The high-resolution structures of the human NK1 receptor (NK1R) bound to small-molecule antagonist therapeutics has been solved recently by X-ray crystallography and NMR spectroscopy (Schöppe et al, 2019; Chen et al, 2019). Here, the NK1R serves as a test case for comparing to cryo-EM structures of different streptavidin-(NK1R-halotag)ₙ constructs, with n=1-4, with those of the published NMR and X-ray structures.

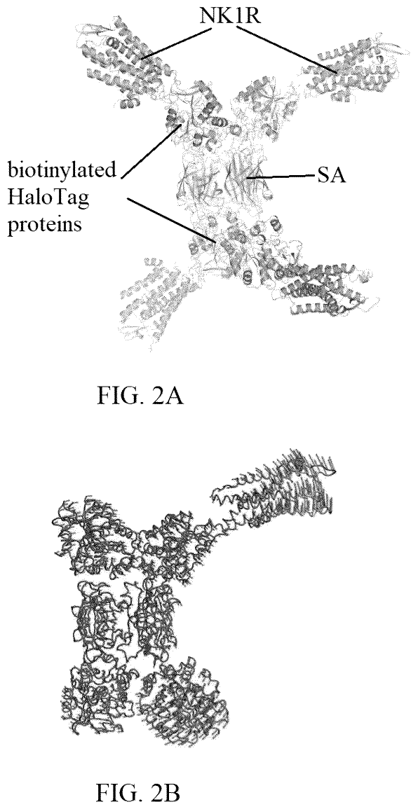

Molecular modeling is a very important step in finding a proper position of the biotinylated tag or the strep tag in the protein sequence of the GPCR. In a first step, homology computer modeling will be used to establish a 3D structural model of any GPCR whose structure has to be solved experimentally by the new cryo-EM approach. In a second step, further computer modeling will be used to insert the biotinylated tag of interest on one of the positions of the GPCRs outlined in FIGS. 1A-1C. The exact insertion within the protein sequence will be adjusted by computer modeling, to obtain optimal structural rigidity between the streptavidin or streptactin template and the GPCR, if necessary by inserting additional spacer amino acids between the original GPCR amino acid sequence and that of the tag or removing flexible amino acid sequences which are functionally not relevant. Molecular dynamics simulations will be used to probe the flexibility/rigidity of the target protein with respect to the streptavidin template. These simulations will help to optimize the position of the biotin at the target protein to reach best accessibility for binding streptavidin paired with lowest mobility. Examples are given for the (HaloTag-biotin)-streptavidin system in FIGS. 2A-2B.

FIG. 2A shows a structural model of SA-(NK1R-halotag)₄, comprising tetrameric streptavidin, SA (center), and four bound molecules of biotinylated HaloTag proteins, each one inserted in a third intracellular loop I3 (referring to FIG. 1A), and amino acid sequence between amino acid 227 and 237 is removed from the NK1 receptor.

FIG. 2B shows intrinsic mobility of the protein complex calculated from anisotropic network model (ANM) analysis. The green vector lengths correlate with the amplitudes of the relative motions of the different protein domains. There is an overall movement of each of the NK1 receptor subunits relative to the individual HaloTag proteins relative to the SA template. Depicted is only one NK1 receptor incorporated into a POPC lipid bilayer, which is not shown for clarity.

It is obvious that the method described before can be easily adapted to solve the structure of water-soluble proteins as targets.

### Example 1

### Computer Modelling

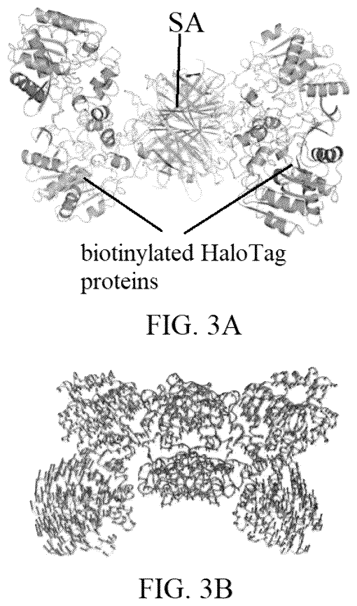

FIG. 3A shows a structural model of SA(HaloTag)₄, comprising tetrameric streptavidin, SA, (center, orange color) and four bound molecules of biotinylated HaloTag proteins (green color), one HaloTag protein per SA subunit.

FIG. 3B shows an intrinsic mobility of complex calculated from ANM analysis. The green vector lengths correlate with the amplitudes of the motions of the protein domains. There is an overall movement of the HaloTag proteins relative to the SA template. ANM is a computational tool for the analysis of internal motions in molecular structures. Details of computational methods for building structural models and calculating internal motions are described as follows.

All models were built with the 3D builder tool in Schrodinger Maestro software. The ionization states of protein were assigned properly according to the results from Schrodinger. MD simulations were carried out using the Desmond software. The optimized potentials for the liquid simulations (OPLS)-3e force field were used in this system, the protein was solvated with the simple point charged (TIP3P) water model and 0.15 M NaCl. The orthorhombic water box was used to create a 15 Åbuffer region between the protein atoms and box sides. The temperature was maintained constant at 310 K, and a 2.0 fs value was obtained in the integration step. We investigated the structure by performing 50 ns unbiased molecular dynamics (MD) analysis, and ANM calculation was utilized to analyze the protein backbone motion in MD trajectory.

### Example 2

### Halotag PEG-Biotin Ligands Synthesis

A HaloTag-PEG4-biotin ligand was synthesized. The HaloTag-PEG4-biotin ligand has a molecular formula of C₃₁H₅₇ClN₄O₉S, a molecular mass of 697, and the following chemical structure:

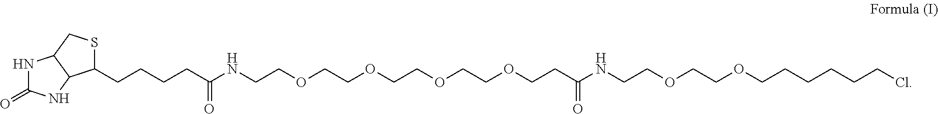 [SMILES: O=C(CCCCC1SCC2NC(=O)NC21)NCCOCCOCCOCCOCCC(=O)NCCOCCOCCCCCCCl]

A terminal —Cl of the HaloTag-PEG4-biotin ligand is configured to be displaced in a nucleophilic reaction by an Asp106 of the HaloTag protein to form the stable ester bond (Los et al, 2008), whereby forming the biotinylated Halo-Tag.

2-(2-((6-Chlorohexyl)oxy)ethoxy)ethanamine hydrochloride and NHS-PEO4-Biotin were used to synthesis the PEG-Biotin Ligands. To a stirring solution of NHS-PEO4-Biotin (5 mg, 8.5×10⁻⁶ mol) in 115 μl dry DMF was added via syringe a 0.3 M solution of 2-(2-((6-Chlorohexyl)oxy)ethoxy)ethanamine hydrochloride (85 μl, 2.55×10⁻⁵ mol) in CH₂Cl₂ followed by one drop of diisoproplyethylamine (excess). The reaction mixture was stirred for 4 hours, then diluted to 1 ml of water and subjected to preparative HPLC purification. H₂O and acetonitrile were used as solvent for preparative HPLC and optical absorbance was detected at 210 nm. Purified sample was analysed on LC-MS using H₂O-formic acid (1%) and acetonitrile as solvent.

A main chemical reaction formula is as follows:

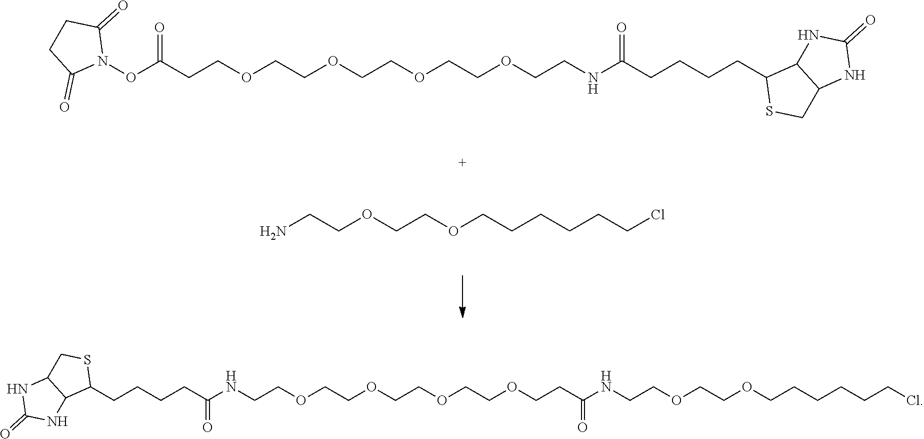 [SMILES: O=C(CCCCC1SCC2NC(=O)NC21)NCCOCCOCCOCCOCCC(=O)ON1C(=O)CCC1=O ; NCCOCCOCCCCCCCl ; O=C(CCCCC1SCC2NC(=O)NC21)NCCOCCOCCOCCOCCC(=O)NCCOCCOCCCCCCCl]

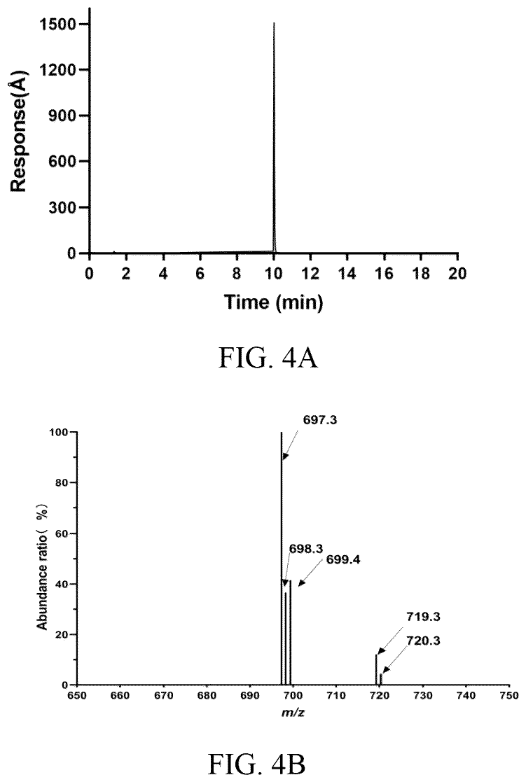

FIG. 4A shows purified HaloTag-PEG4-biotin ligand analyzed by HPLC: the absorption wavelength was 210 nm and the retention time 10 mins.

FIG. 4B shows LC-MS of HaloTag-PEG4-biotin ligand. MS: m/z calcd. for C₃₁H₅₈ClN₄O₉S⁺: 697.36 (100%), 698.36 (36.6%), 699.36 (41%), 719.3 (12.13%), 720.3 (4.41%).

### Example 3

### Expression and Purification of Halotag Protein

The DNA fragments of wild type halotag protein were amplified by PCR and cloned into a pET29a vector. For protein production, the plasmids were transformed into Escherichia coli BL21 (DE3) cells. The cells were grown at 37° C. until an OD600=0.6 was reached, then the sample was shifted to 18° C., and proteins were induced by adding 0.2 mM isopropyl β-D-1-thiogalactopyranoside (IPTG). Cells were harvested using centrifugation after 18 hours post induction and resuspended in a lysis buffer comprising 25 mM Tris-HCl pH 7.5, 400 mM NaCl and 10 mM imidazole. Cells were lysed by passing the cell suspension through a French press (800 p.s.i.). The lysed cell preparation was centrifuged for 20 min at 12,000×g. The pellet was discarded and the supernatant was applied onto nickel affinity column equilibrated with the binding buffer consisting of 25 mM Tris-HCl, pH 7.5, 400 mM NaCl and 10 mM imidazole. The proteins bound on the column resin were first washed with 50 mM imidazole and then eluted with 250 mM imidazole. The obtained protein preparation was dialyzed twice against 25 mM Tris-HCl (pH 8.0), 100 mM NaCl. Finally, size exclusion chromatography was used for isolating the monomeric form of the halotag protein.

### Example 4

### Size Exclusion Chromatography of Streptavidin-Halotag Protein Complex

The streptavidin (purchased from the IBA, Gottingen, Germany) was mixed with the halotag-PEG4-biotin ligand at the molar ration of 1:4. After 3 hours incubation, monomer of halotag protein was added to the mixture. After overnight incubation, the mixture of streptavidin, halotag-PEG4-biotin ligand and halotag protein was applied to size exclusion chromatography to separate the formed streptavidin-halotag protein complex from the reaction mixture.

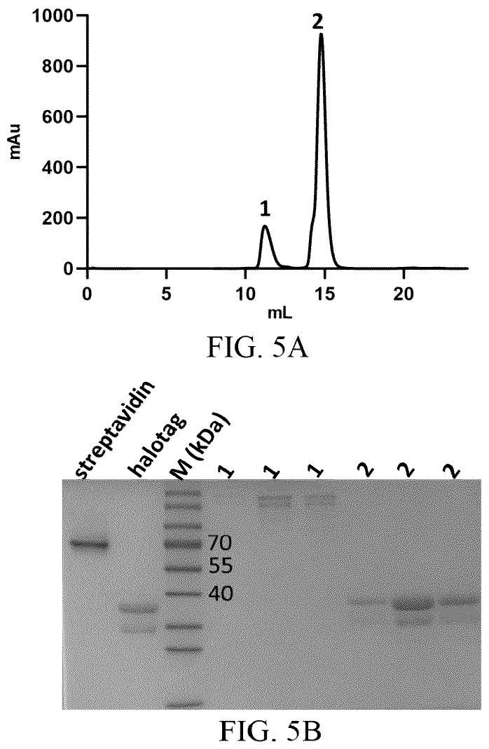

FIG. 5A shows a size exclusion chromatography of streptavidin-halotag protein complex. FIG. 5B shows SDS-PAGE of streptavidin tetramer (indicated as streptavidin), halotag protein (indicated as halotag), size exclusion chromatography fraction 1 and fraction 2 (FIG. 5A). Size exclusion chromatography and SDS-PAGE indicate that the streptavidin-halotag protein complex was formed.

### Example 5

### Electron Micrographs of Negative Stained Streptavidin-Halotag Protein Complex

Electron microscopy of negative stained samples were used to evaluate the protein quality. In brief, 3 μL of freshly purified streptavidin-halotag protein complex (0.025 mg/ml) was applied onto copper grids supported by a thin layer of glow-discharged carbon film (Zhongjingkeyi Technology Co., Ltd). After adsorption for 1 min, uranyl acetate (2% w/v) was used for negative staining at room temperature. The negative stained grid was examined using FEI Talos L120C operated at 120 kV.

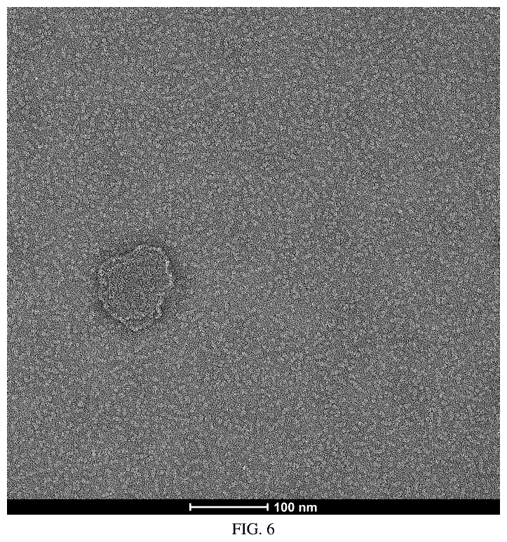

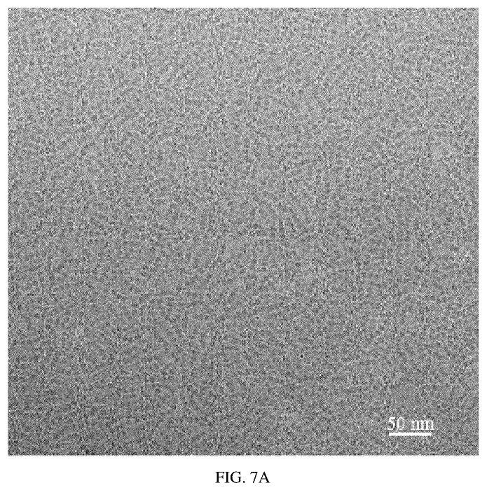

FIG. 6 shows a representative electron micrograph of a negative stained preparation of the streptavidin-halotag protein complex isolated by size exclusion chromatography (corresponding to fraction 1 shown in FIG. 5A).

### Example 6

### Preparation of Grids for Cryo-EM

Peak fractions collected from the size exclusion chromatography were concentrated to 1 mg/mL and then centrifuged at 12,000×g for 30 min at 4° C. A total of 4 μL of sample was applied to glow-discharged Quantifoil porous carbon grids (gold, 2-2 μm/well, 300 mesh), blotted using a Vitrobot (FEI) with a blotting time of 2 seconds at a temperature of 4° C. at 100% humidity, and then frozen in liquid nitrogen cooled liquid ethane. Frozen grids were carefully transferred and stored in liquid nitrogen until cryo-EM images were collected.

### Example 7

### Data Acquisition and Image Processing of Cryo-EM

A total of 19060 movies were collected on a Titan Krios G3i (Thermofisher Scientific) operated at 300 kV equipped with a Gatan image filter Continuum 1069 (operated with a slit width of 20 eV), mounted with a K3 Summit detector (Gatan, Inc.). EPU was used to automatically acquire micrographs in super-resolution counting mode at a pixel size of 0.4275 Å and with nominal defocus values ranging from −1.5 to −2.5 μm. Movies with 32 frames each were collected at a dose of 50 electrons per pixel per second over an exposure time of 2.1 s, resulting in a total dose of 50 e⁻/Å² on the specimen. Tilting data at 10°, 20°, 30° and 40° were collected to overcome the preferred orientation issue of the streptavidin-halotag complex. All movie frames in each stack were aligned and dose weighted using MotionCor2, which generated 2-fold binned images to a pixel size of 0.855 Å/pixel. The patch ctf module of Cryosparc2 software was used for estimating the defocus values and astigmatism parameters of the contrast transfer function (CTF). A total of 12655 micrographs were chosen for further processing. To investigate the subunit organization, reference-free two-dimensional (2D) classifications were performed. The 2D classification module of Cryosparc2 was used for further data processing. 5850507 particles were initially picked from selected micrographs and subjected to 2D classification. After performing 2D classification in Cryosparc, the best-looking 2D class averages, as judged by visual inspection, were selected to build an ab initio reconstruction for heterogeneous refinement in cryoSPARC. After heterogeneous refinement, one class showing intact features (particles) was selected and subjected to nonuniform refinement with C2 symmetry, followed by local refinement, yielding an overall resolution of 3.6 Å.

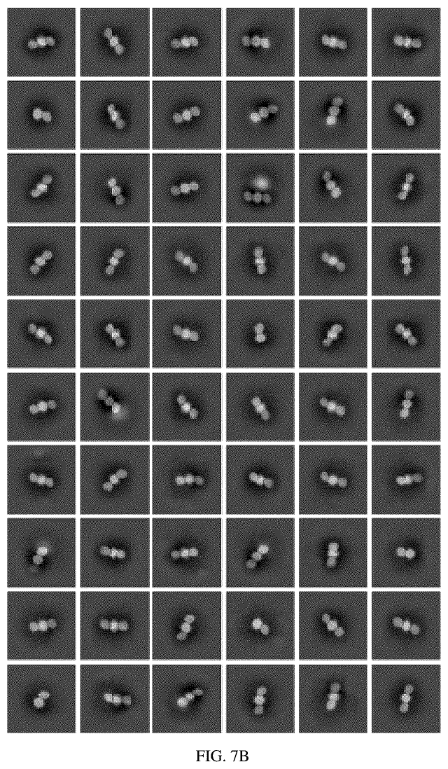

FIGS. 7A-7B show representative micrograph and 2D classification of streptavidin-halotag complex. FIG. 7A is a micrograph recorded using a cryo-electron microscope operated at 300 kV. Scale bar: 50 nm. FIG. 7B is representative 2D class-averaged images of the complex.

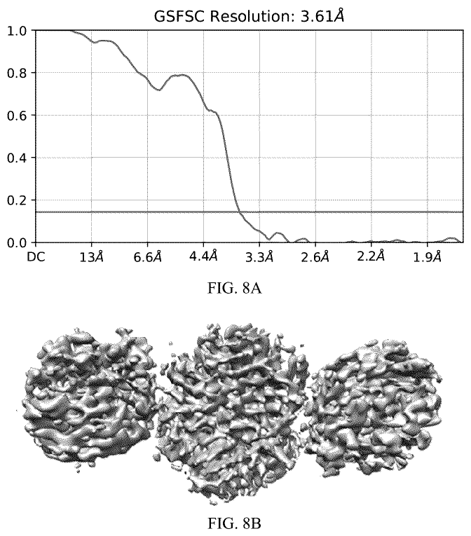

FIGS. 8A-8B. Resolution estimation of cryo-EM map. Particularly, FIG. 8A shows Fourier shell correlation (FSC) plots indicating corrected resolutions as 3.6 Å; and FIG. 8B depicts a 3D map of streptavidin-halotag complex after non-uniform refinement.

### Example 8

### Expression and Purification of NK1R-Halotag Fusion Protein

The DNA coding sequence of the Halotag protein was inserted into the intracellular loop 3 of the coding sequence of the NK1R and then the construct was amplified by PCR and cloned into a pEG BacMam vector. HEK293F cells were cultured in SMM 293T-II medium under 8% CO2 in an incubation shaker at 37° C. Target protein was expressed by transient transfection of the HEK293F cells using plasmid DNA coding for the NK1R-halotag fusion protein. Briefly, for 1 liter culture of HEK293F cells, 1 mg plasmid DNA and 4 mg 25-kDa linear polyethylenimines were preincubated for 10 min in 25 ml fresh medium, and then two media samples were mixed for further incubation for 15 min prior to adding the mixture to cells. The transfected cells were cultured for 72 h before harvest by centrifugation. For each batch of protein purification, 4 liters of transfected HEK293F cells were harvested by centrifugation at 2000×g. Cell pellets were resuspended in lysis buffer containing 20 mM HEPES, pH 7.5, 500 mM NaCl, 1 mM MgCl₂, 1 mM CaCl2, 0.1 mM PMSF and 1×protease inhibitor cocktail and disrupted by French press. Cell membranes were solubilized in lysis buffer containing 1% (wt/vol) N-dodecyl-β-D-maltoside (DDM), 0.2% Cholesteryl Hemisuccinate Tris Salt (CHS) and 0.5 μM netupitant for 12 h with stirring at 4° C. Solubilized NK1R-halotag protein was separated from the insoluble fraction by centrifugation for 1 h at 40,000×g and incubated with 2 ml of TALON metal affinity resin for 3 h at 4° C. The resin was then washed with 10 column volumes of buffer containing 20 mM Tris pH 7.5, 150 mM NaCl, 0.5 μM netupitant, 0.1 mM PMSF and 0.1% DDM, 0.02% CHS and 30 mM imidazole. Resin was eluted by 5 column volumes of buffer containing 20 mM Tris pH 7.5, 150 mM NaCl, 0.5 μM netupitant, 0.1 mM PMSF and 0.1% DDM, 0.02% CHS and 250 mM imidazole. The eluted protein was collected and concentrated using a 100 kDa concentrator. The streptavidin (purchased from IBA, Gottingen, Germany) was mixed with the Halotag-PEG4-biotin ligand at the molar ration of 1:4 to bind four Halotag-PEG4-biotin ligands to one streptavidin tetramer, named SA(Halotag PEG-Biotin ligand)ₙ (n=1-4). After 3 hours incubation, NK1R-Halotag fusion protein was added to the mixture to covalently bind to the halotag ligand of SA(Halotag PEG-Biotin ligand)ₙ. After overnight incubation, the reaction mixture was applied to size exclusion chromatography to separate the formed streptavidin-NK1R-halotag complex from the reaction mixture.

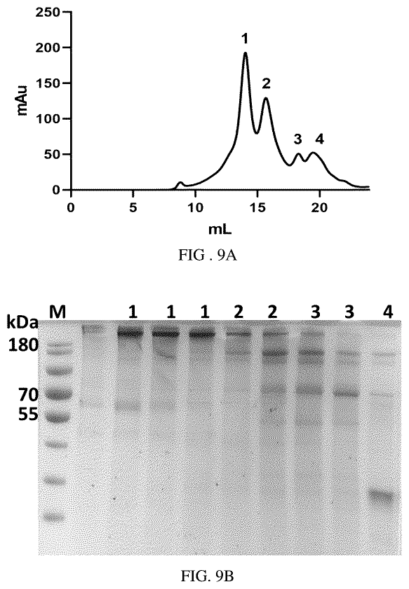

FIG. 9A shows a size exclusion chromatography of streptavidin-NK1R-halotag complex. FIG. 9B shows SDS-PAGE of streptavidin NK1R-halotag complex, NK1R-Halotag, size exclusion chromatography peak 1 to peak 4 (FIG. 9A). Size exclusion chromatography and SDS-PAGE indicate the streptavidin NK1R-halotag complex was formed.

The terms “comprising,” “having,” “including,” and “containing” are to be construed as open-ended terms (i.e., meaning “including, but not limited to,”) unless otherwise noted. While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

### REFERENCES

Adachi N et al (2019). Differential S-palmitoylation of the human and rodent □3-adrenergic receptors. J Biol Chem 294:2569-2578. https://doi.org/10.1074/jbc.RA118.004978.
Anbar R and Bouvier M (2003). Role of palmitoylation/depalmitoylation reactions in G-protein-coupled receptor function. Pharmacol & Therapeut 97:1-33. DOI: 10.1016/s0163-7258(02)00300-5.
Baidya M et al (2020). Genetically encoded intrabody sensors report the interaction and trafficking of -arrestin 1 upon activation of G-protein-coupled receptors. J Biol Chem 295: 10153-10167. DOI: https://doi.org/10.1074/jbc.RA120.013470.
Bieri et al, NATURE BIOTECHNOLOGY VOL 17 NOVEMBER 1999.
Chan Hyuk Kim, Jun Y Axup and Peter G Schultz. Protein conjugation with genetically encoded unnatural amino Acids. Current Opinion in Chemical Biology 2013, 17:412-419.
Chen Q et al (2021). Structures of rhodopsin in complex with G-protein-coupled receptor kinase 1. Nature 595:600-605. https://doi.org/10.1038/s41586-021-03721-x.
Chen S et al (2019). Human substance P receptor binding mode of the antagonist drug aprepitant by NMR and crystallography. Nat Commun 10:638.
   https://doi.org/10.1038/s41467-019-08568-5.
D'Imprima E and Kühlbrandt W (2021). Current limitations to high-resolution structure determination by single-particle cryoEM. Quarterly Reviews of Biophysics, Volume 54, 2021, e4. DOI: https://doi.org/10.1017S0033583521000020
Fairhead M & Howarth M. (2015). Site-specific biotinylation of purified proteins using BirA. Meth Mol Blot 1266:171-184. https://link.springer.com/protocol/10.1007%2F978-1-4939-2272-7_12.
Garcia-Nafria J et al (2020). Cryo-electron microscopy: Moving beyond X-ray crystal structures for drug receptors and drug development. Annu Rev Pharmacol Toxicol. 60:51-71. https://doi.org/10.1146/annurev-pharmtox-010919-023545
George N et al (2004). Specific labeling of cell surface proteins with chemically diverse compounds. J Am Chem Soc 126:8896-8897. PMID:15264811
Greber B J et al (2021). 2.5 A-resolution structure of human CDK-activating kinase bound to the clinical inhibitor ICEC0942. Biophys J 120, 677-686.
   https://doi.org/10.1016/j.bpj.2020.12.030.
Külbrandt W (2014). The resolution revolution. Science 343:1443-1444. DOI: 10.1126/science.1251652
Lee et al (2020). Molecular basis of beta-arrestin coupling to formoterol-bound betal-adrenoceptor. Nature 583:862-866 https://doi.org/10.1038/s41586-020-2419-1.
Li F et al (2021). Highlighting membrane protein structure and function: A celebration of the Protein Data Bank. J Blot Chem 296:100557.
   https://doi.org/10.1016/j.jbc.2021.100557.
Lin K et al (2020). A simple method for non-denaturing purification of biotin-tagged proteins through competitive elution with free biotin. BioTechniques 68:41-44. DOI: 10.2144/btn-2019-0088.
Los G V et al (2008). Halolag: a novel protein labeling technology for cell imaging and protein analysis. ACS Chem Biol 3:373-82. doi:10.1021/cb800025k. PMID 18533659.
Manglik A et al (2017). Nanobodies to Study G-Protein-Coupled Receptor Structure and Function. Annu Rev Pharmacol Toxicol 57:19-37. DOI: 10.1146/annurev-pharmtox-010716-104710.
Patwardhan A et al (2021). Post-Translational Modifications of G Protein-Coupled Receptors Control Cellular Signaling Dynamics in Space and Time. Pharmacol Rev 73:120-151. https://doi.org/10.1124/pharmrev.120.000082.
Purskow J A et al (2020). NMR Methods for Structural Characterization of Protein-Protein Complexes. Front Mol Biosci 7, Article 9.
   https://doi.org/10.3389/fmolb.2020.00009.
Reymond et al (2011). Visualizing Biochemical Activities in Living Cells through Chemistry. Chimia 65:868-871. doi:10.2533/chimia.2011.868.
Schmidt T G M and Skerra A (2007). The Strep-tag system for one-step purification and high-affinity detection or capturing of proteins. Nature Protocols 2:1528-1535.
   doi:10.1038/nprot.2007.209. PMID 17571060.
Schöppe J et al (2019). Crystal structures of the human neurokinin 1 receptor in complex with clinically used antagonists. Nat Commun 10:17. https://doi.org/10.1038/s41467-018-07939-8.
Sriram K and Insel P A (2018). G Protein-Coupled Receptors as Targets for Approved Drugs: How Many Targets and How Many Drugs? Mol Pharmacol 93:251-258. DOI: https://doi.org/10.1124/mol.117.111062.
Vivero-Pol L et al (2005). Multicolor imaging of cell surface proteins. J Am Chem Soc 127:12770-1. PMID:16159249.
Zhou Z et al (2007). Genetically Encoded Short Peptide Tags for Orthogonal Protein Labeling by Sfp and AcpS Phosphopantetheinyl Transferases. ACS Chem Biol 2:337-346.https://doi.org/10.1021/cb700054k.
Zhou Q et al (2019). Common activation mechanism of class A GPCRs. eLife 8:e50279. DOI: https://doi.org/10.7554/eLife.50279.

What is claimed is:

1. A method for determining a protein structure using cryo-electron microscopy, the method comprising:
enabling a target protein to contain a tag;
enabling a resulting target containing the tag to bind a scaffold protein to form a complex between the target protein and the scaffold protein; and
performing single-particle imaging using the cryo-electron microscopy to determine a structure of the target protein in complex with the scaffold protein;
wherein
the scaffold protein is any one of streptavidin, avidin, or derivatives thereof;
the tag is configured for selectively binding to the scaffold protein; and
the tag is one selected from the group consisting of:
a biotin tag, comprising a biotin;
a biotinylated protein or polypeptide tag, comprising a protein sequence and a biotin covalently linked to the protein sequence;
a Strep-tag; and
a biotinylated or strep-tagged antibody, or antibody Fab fragment, or single-chain antibody.

2. The method according to claim 1, wherein
in case that the tag is the biotin tag, the step of enabling the target protein to contain the tag comprises:
enabling the biotin tag to be contained in a side chain of a non-canonical amino acid, and site-specifically introducing the non-canonical amino acid into an amino acid sequence of the target protein;
or alternatively, chemically attaching the biotin tag to a specific side chain of an amino acid and site-specifically introducing the amino acid to the target protein;
or alternatively, chemically attaching the biotin tag to a specific glycosylation site at the N-terminal part of the target protein.

3. The method according to claim 1, wherein
the biotinylated protein or polypeptide tag is one selected from the group consisting of:
a self-labeling protein tag, which allows site-specific covalent attachment of a biotin residue to a respective tag protein;
an acyl carrier protein tag (ACP-tag) or a peptidyl carrier protein tag (PCP-tag); and
an Avi-tag, adapted to be fused to an N-terminus, a C-terminus, or an exposed loop region of the target protein, and to be covalently attached to the biotin using Escherichia coli biotinylase BirA.

4. The method according to claim 3, wherein the self-labeling protein tag is a Snap-tag, a Clip-tag, or a Halo-tag.

5. The method according to claim 1, wherein the target protein has a molecular mass between 50 kDa and 20 kDa.

6. The method according to claim 1, wherein the target protein is a water-soluble protein or a membrane protein.

7. The method according to claim 1, wherein in the case that the target protein is a G protein-coupled receptor (GPCR), the biotin tag or the biotinylated protein or polypeptide tag is inserted to the N-terminus, the C-terminus, one of the extracellular loops, or one of the intracellular loops of the GPCR.

8. The method according to claim 1, wherein in the case that the target protein is a G protein-coupled receptor (GPCR), to which an anticalin or an Ig type or single-chain antibody is selectively bound to the N-terminus, the C-terminus, one of extracellular loops, or one of intracellular loops of the GPCR.

9. The method according to claim 7, wherein the biotin tag or the biotinylated protein or polypeptide tag or a Strep-tag is fused into an intracellular loop I4 of the GPCR, so as to stabilize a bound streptavidin or streptactin in a rigid structure.

10. The method according to claim 7, wherein the biotin tag or the biotinylated protein or polypeptide tag or the Strep-tag is fused into one of extracellular loops or one of intracellular loops of the GPCR, so as to stabilize a bound streptavidin or streptactin in a rigid structure.

11. The method according to claim 1, wherein in the case that the target protein is a complex of a prototypical GPCR and an intracellular signaling protein, the tag is introduced by adopting any one of the following manners, so as to enable binding of the complex to the streptavidin or the derivative thereof as the scaffold protein:
1) inserting the biotin tag or the biotinylated protein or polypeptide tag to the N-terminus, the C-terminus, one of extracellular loops, or one of intracellular loops of the prototypical GPCR;
2) selectively binding an anticalin or an Ig type or single-chain antibody to the N-terminus, the C-terminus, one of extracellular loops, or one of intracellular loops of the prototypical GPCR;
3) fusing the biotin tag or the biotinylated protein or polypeptide tag to the intracellular signaling protein sequence;
4) selectively binding a biotinylated antibody to the intracellular signaling protein; or
5) selectively binding a biotinylated anticalin to the intracellular signaling protein.

12. The method according to claim 11, wherein the intracellular signaling protein is any one selected from the following:
a heterotrimeric G-protein or a mini-G-protein, both of which are adapted to bind to an agonist-activated GPCR;
a G-protein-coupled receptor kinase (GRK), adapted to bind to and phosphorylates an active GPCR;
an arrestin, adapted to bind to a phosphorylated GPCR; and
a peptide sequence, mimicking a region of the G-protein or the arrestin for receptor binding.

13. The method according to claim 1, wherein in the case that the target protein is a neurokinin 1 receptor (NK1R), the tag is a biotinylated Halo-tag, the scaffold protein is streptavidin; a tetrameric NK1R is assembled on the streptavidin via the biotinylated Halo-tag, whereby forming a complex SA(HaloTag-NK1R)ₙ, wherein n is an integer ranged between 1 and 4.

14. The method according to claim 13, wherein the biotinylated Halo-tag is inserted into a third intracellular loop IL3 of the NK1R, and a sequence of between amino acid 227 and 237 is removed from the third intracellular loop.

15. The method according to claim 13, before the step of enabling the target protein to contain the tag, further comprising:
synthesizing a HaloTag-PEG4-biotin ligand, and
forming a stable ester bond between the HaloTag-PEG4-biotin ligand and a HaloTag protein whereby forming a biotinylated Halo-Tag.

16. The method according to claim 15, wherein the HaloTag-PEG4-biotin ligand has a molecular formula of C₃₁H₅₇ClN₄O₉S, a molecular mass of 697, and the following chemical structure: 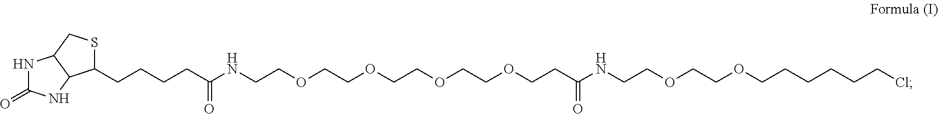 [SMILES: O=C(CCCCC1SCC2NC(=O)NC21)NCCOCCOCCOCCOCCC(=O)NCCOCCOCCCCCCCl]
wherein
a terminal —Cl of the HaloTag-PEG4-biotin ligand is configured to be displaced in a nucleophilic reaction by an Asp106 of the HaloTag protein to form the stable ester bond, whereby forming the biotinylated Halo-Tag.

17. The method according to claim 1, before the step of enabling the target protein to contain the tag, further comprising: molecular modeling,
wherein
a proper position in the target protein suitable for inserting the biotin tag or the biotinylated protein or polypeptide tag or the Strep-tag is found and adjusted, to obtain an optimal structural rigidity between the scaffold protein and the target protein.

18. The method according to claim 17, wherein during the molecular modeling, spacer amino acids are inserted into an amino acid sequence of the target protein, or flexible amino acid sequences which are functionally not relevant are removed from the amino acid sequence of the target protein.

19. The method according to claim 16, wherein a PEG4 spacer in the HaloTag-PEG4-biotin ligand is further shortened or enlarged to obtain an optimal structural rigidity between the scaffold protein and the target protein.
